(19) United States
(12) Patent Application Publication
(10) Pub. No.: US 2023/0201256 A1
(43) Pub. Date: Jun. 29, 2023

(54) CALCINEURIN INHIBITOR TO IMPROVE CD3+CELL SURVIVAL TO THEREBY FACILITATE ENGRAFTMENT OF DONOR CD34+ CELLS IN A RECIPIENT

(71) Applicant: MEDEOR THERAPEUTICS, INC., South San Francisco, CA (US)

(72) Inventor: Giovanni Ferrara, South San Francisco, CA (US)

(73) Assignee: MEDEOR THERAPEUTICS, INC., South San Francisco, CA (US)

(21) Appl. No.: 17/926,268

(22) PCT Filed: May 19, 2021
(86) PCT No.: PCT/US2021/033065

§ 371 Date: Nov. 18, 2022

Related U.S. Application Data
(60) Provisional application No. 63/028,215, filed on May 21, 2020.

(51) Int. Cl. A61K 35/17; A61K 35/22; A61K 38/13; A61K 31/436

(52) U.S. Cl. CPC A61K 35/17; A61K 35/22; A61K 38/13; A61K 31/436

(57) ABSTRACT

Provided are improved methods for grafting donor derived CDS 4+ cells in an organ transplant recipient comprising administration CD3+ cells together with a calcineurin inhibitor in an effective amount to reduce or prevent an immune system of the recipient from rejecting the CD3+ cells from the donor, thereby enabling the CD3+ cells of the donor to facilitate engraftment of the CDS 4+ cells from the donor. In certain embodiments, the effective amount of the calcineurin inhibitor provided to reduce or prevent the immune system of the recipient from rejecting the CD3+ cells of the donor is lower than an amount provided for protecting the organ of the donor from rejection by the immune system of the recipient.

### CROSS-REFERENCE TO RELATED APPLICATION

This application claims the benefit of, and priority to, U.S. Provisional Patent Application No. 63/028,215, filed May 21, 2020, the contents of which are incorporated by reference.

### FIELD OF THE INVENTION

The invention generally relates to methods of using a calcineurin inhibitor to improve survival of donor CD3+ cells in a recipient, which thereby facilitates engraftment of CD34+ cells from the donor in the recipient.

### BACKGROUND

Advances in surgical techniques and improved drugs that prevent infection and rejection have allowed transplantation of organs to become an effective treatment for many diseases. Transplanted organs include heart, intestine, liver, lung, pancreas and kidney, as well as non-solid organs such as hematopoietic tissue. The major barrier to organ transplantation between genetically non-identical patients lies in the recipient's immune system, which can respond to the transplanted organ as “non-self” and reject it. Consequently, most transplant recipients must take immunosuppressive drugs prior to receiving a transplanted organ. Recipients usually receive a mixture of three maintenance immunosuppressive drugs, including a calcineurin inhibitor such as cyclosporine A, tacrolimus or sirolimus; prednisone; and an inhibitor of nucleic acid synthesis such as mycophenolate mofetil. Immunosuppressive drugs, however, place the recipient at greater risk of infection and cancer, in addition to the side effects of the medications, such as hypertension, nephrotoxicity, infection, and heart disease. Moreover, in spite of modern immunosuppressive drugs, in some centers acute rejection can still occur in 10-25% of people after transplant. As a result, generally transplant recipients will take immunosuppressive anti-rejection drugs for as long as the transplanted organ remains with the host, whether or not the organ continues to function. Even for mixtures of widely used immunosuppressives, the cost can be high.

Clinical data from the last decade has shown that long-term graft tolerance in humans without immunosuppression can be achieved by reconstructing the recipient's immune system using donor-derived or recipient-derived hematopoietic cellular compositions that include hematopoietic stem and progenitor cells (HSPCs) and T cells, such as CD34+ and CD3+ cells. Immune system engraftment confers several advantages on organ transplant recipients. First, it improves the recipient's chances of tolerating the graft. In addition, it allows transplant recipients to discontinue immunosuppressive therapy and their attendant several deleterious side effects.

Unfortunately, the supply of blood cells from adult donors is limited by the difficulties of obtaining hematopoietic cells from donors and the quantity of blood cells that can be collected from recipients prior to the administration of immunosuppressive therapy. It is therefore of great clinical interest to develop improved methods for the engraftment hematopoietic cells including CD34+ cells and achieve tolerance and complete withdrawal from immunosuppressive drugs in adult transplant patients while maximizing available quantities of hematopoietic cells that can be recovered from a donor or from the recipient.

### SUMMARY

The invention recognizes that calcineurin inhibitors, previously known to be useful in specific dosages as an immunosuppressant, can be used at lower dosages after organ transplantation to improve the engraftment of donor derived hematopoietic cellular compositions comprising CD34+ cells when given in combination with CD3+ cells. This is accomplished by maintaining the survival and function of donor CD3+ cells, which can in turn render the host bone marrow environment more amenable to engraftment of CD34+ cells. Accordingly, provided is a method for transplantation of solid organs and non-solid organs from a donor comprising implanting the human organ in a recipient human body, administering to the recipient a composition comprising donor derived CD34+ and CD3+ cells, and administering to the recipient a regimen comprising a calcineurin inhibitor in an effective amount to maintain survival and function of the CD3+ cells in the recipient. Advantageously, the effective amount of the calcineurin inhibitor provided to improve survival and function of the CD3+ cells is lower than an amount provided for immune suppression therapy. Without being limited to a single mechanism, it has been discovered that this lower effective amount of the calcineurin inhibitor counteracts host CD3+ cells allowing for a more amenable environment for the survival and function of donor derived CD3+ which protect and thereby facilitate the engraftment and proliferation of donor derived CD34+ cells.

Compositions of the present invention comprise donor derived CD3+ cells together with donor derived CD34+ cells. Advantageously, CD3+ cells promote engraftment of the CD34+ cells. Compositions comprising CD3+ cells and CD34+ cells may also comprise additives which promote engraftment of the CD3+ cells. Compositions comprising the CD3+ cells and CD34+ cells can include various concentrations for each of the CD34⁺ cells and CD3⁺ cells, and different concentrations are discussed herein. The amount may be specified as a number of cells relative to the body mass of the recipient. For example, the cellular product may contain at least 1×10⁵, 2×10⁵, 5×10⁵, 1×10⁶, 2×10⁶, or 4×10⁶ CD34⁺ cells/kg recipient weight. The cellular product may contain at least 1×10⁴, 2×10⁴, 5×10⁴, 1×10⁵, 2×10⁵, 5×10⁵, 1×10⁶, 2×10⁶, 5×10⁶, 1×10⁷, 2×10⁷, 5×10⁷, or 1×10⁸ CD3⁺ cells/kg recipient weight.

Compositions comprising CD3+ and CD34+ cells accordingly to aspects of the invention are described in U.S. Pat. Nos. 8,506,954; 9,114,157; 9,192,627; 9,290,813; 9,504,715; 9,504,717; 9,545,444; 9,561,253; 9,833,477; 9,974,807; 10,076,542; 10,080,769; 10,159,694; 10,166,256; 10,183,043; 10,256,648; 10,286,049; 10,549,082; 10,603,340; U.S. Patent Application Publication Nos. US 2016-0376653; US 2018-024337; US 2019-0083530; US 2019-0083537; US 2019-0091262; US 2019-0192561; US 2019-0192562; US 2019-0201514; US 2019-0275085; US 2019-0298762; US 2019-0307803; US 2019-0307804; US 2019-0336528; US 2019-0358257; US 2019-0307803; US 2020-0086004; US 2020-0087627; PCT International Application Publication Nos. WO/2012/096974; WO/2014/035695; WO/2014/133729; WO/2014/172532; WO/2019/178106; WO/2019/195657; WO/2019/195659; WO/2020/061180; PCT International Application No. PCT/US2019/051486; U.S. patent application Ser. Nos. 16/109,373; 16/573,387; 16/573,395; 16/573,408; 16/573,420; 16/716,102; and U.S. Provisional Application No. 62/858,142, the contents of each of which are herein incorporated by reference.

Compositions comprising CD3+ according to aspects of the present invention may also comprise hematopoietic facilitatory or human facilitating cells (hFCs). Advantageously the hFCs may be selected and provided to improve engraftment of the CD34+ cells. The compositions may comprise CD3+ cells and CD34+ cells, or CD3+ cells, CD34+ cells, and hFCs. CD3+ cells and CD34+ cells may be provided together in a single composition and the hFCs provided in a separate composition, or the CD3+ cells, CD34+ cells, and hFCs, may be provided together. hFCs are generally characterized as CD8+ and alpha beta TCR−. CD8+/alpha beta TCR hFCs may be CD8+/alpha beta TCR−/CD56^{dim/neg} or may be CD8+/alpha beta TCR−/CD56^{bright}. hFCs may also be characterized by the presence of cells expressing the following markers: CD3 epsilon CD19 , CD11c , CD11b, Foxp3, HLA-DR, and CD123. hFCs may be predominantly CD3 epsilon−/CD19+cells. For example, about 48% of the hFCs may be CD8+/alpha beta TCR−/CD3 epsilon+ cells. In other aspects of the invention, hFCs may be predominantly CD3 epsilon+/CD19− cells. Among CD8+/alpha beta TCR−/CD56^{dim/neg} cells, the majority of cells express CD3 epsilon. Among CD8+/alpha beta TCR−/CD56^{bright} cells, CD3 epsilon is expressed at a much lower level.

Hematopoietic and human facilitating cells and cellular compositions for engrafting CD3+ cells according to aspects of the invention are described in WIPO PCT Publication Nos. WO/1994/001534; WO/1995/018631; WO/1998/026802; WO/1994/001534; WO/1998/026802; WO/1999/026639; WO/2002/040639; WO/2002/040640; WO/2003/012060; WO/2005/001040; WO/2005/040050; WO/2005/023982; WO/2009/148568; and WO/2018/165161; U.S. Pat. Nos. 5,514,364; 5,635,156; 5,772,994; 5,876,692; 6,039,684; 6,217,867; 7,429,376; 8,632,768; and 9,678,062; and U.S. Patent Publication Nos. US 2001-0009663; US 2002-0142462; US 2002-089746; US 2003-0055223; US 2003-0017152; US 2003-0165475; US 2004-0185043; US 2004-0228845; US 2005-0118142; US 2006-0018885; US 2007-0098693; US 2010-0016240; US 2011-0110909; US 2014-0234843, US 2017-0000825; and US 2018-0252729; each of which is herein incorporated by reference.

Any known calcineurin inhibitors may be used in the present invention. For example, cyclosporine A, tacrolimus or sirolimus can be administered to improve survival, tolerance, and/or function of the CD3+ cells. A person having ordinary skill in the art will understand the effective amount of the calcineurin inhibitor is an amount that is effective to improve survival, tolerance, and/or and function of the CD3+ cells while being lower than the amount provided in immunosuppressive therapies. For example, the effective amount of the calcineurin inhibitor may be less than 20 ng/mL, 10 ng/mL, 10 ng/mL, or 5 ng/mL. The effective amount may also be determined be the recipients body weight. For example, the effective amount may be less than 15 mg per kg body weight, 14 mg per kg body weight, 12.5 mg per kg body weight, 6 mg per kg body weight, or less than 2 mg per kg body weight.

The calcineurin inhibitor may be administered via any known route, for example oral administration. The effective amount or dosage of the calcineurin inhibitor will vary based on the route of administration.

In aspects of the invention adjuvant agents may also be combined with a calcineurin inhibitor. Adjuvant agents include steroids, azathioprine, mycophenolic acid (MPA) agents, such as mycophenolate mofetil, mTOR inhibitors, such as sirolimus, and belatacept. For example, the effective amount of the calcineurin inhibitor is administered together with an adjuvant agent selected from the group comprising steroids, azathioprine, mycophenolic acid (MPA) agents, such as mycophenolate mofetil, mTOR inhibitors, such as sirolimus, and belatacept.

The effective amount of the calcineurin inhibitor may also be administered to the recipient simultaneously with administration of the composition comprising CD3+ and CD34+ cells, prior to administration of the composition comprising CD3+ and CD34+ cells, or after administration of the composition comprising CD3+ and CD34+ cells. For example, the calcineurin inhibitor may be administered immediately prior to or immediately after administration of the CD3+ and CD34+ cells. In other aspects of the invention the calcineurin inhibitor is administered prior to administration of the CD3+ and CD34+ cells. When administered prior to administration of the CD3+ and CD34+ cells, the calcineurin inhibitor is administered during a period that the later administration of the CD3+ and CD34+ cells results in improved survival and function of the CD3+ cells, which in turn allows the donor CD3+ cells to facilitate engraftment of the donor CD34+ cells. In other aspects of the invention the calcineurin inhibitor is administered after administration of the cellular product. When administered after administration of the cellular product, the calcineurin inhibitor is administered during a period that improves survival and function of the CD3+ cells. Because survival and function of donor CD3+ cells is improved, the donor CD3+ better facilitate engraftment of donor CD34+ cells.

In aspects of the invention the effective amount of the calcineurin inhibitor is administered about 1 month, about 3 weeks, about 2 weeks, about 1 week, about 5 days, about 3, days, or about 1 day prior to administration of the CD3+ and CD34+ cells. The effective amount of the calcineurin inhibitor may also be administered about 1 month, about 3 weeks, about 2 weeks, about 1 week, about 5 days, about 3, days, or about 1 day after administration of the CD3+ and CD34+ cells. The regimen comprising an effective amount of the calcineurin inhibitor may also be administered for any period necessary to improve engraftment of the CD3+ and CD34+ cells. For examples, the regimen comprising as effective amount of the calcineurin inhibitor may be administered for about 3 days, about 5 days, about 1 week, about 2 weeks, about 3 weeks, about 1 month, about 2 months, or about 3 months, about 4 months, about 5 months, or about 6 months.

Advantageously, the methods of the present invention are effective to reduce the amount, duration, or both the amount and duration of immunosuppressant therapy provided to the recipient after implanting the human organ in the recipient. For example, where an immunosuppressant therapy would have been provided to the recipient following the organ transplant, a reduced amount or no immunosuppressant therapy may be administered. Additionally, because survival and function of the donor CD3+ cells is improved, donor CD34+ cell engraftment is improved, and tolerance established, the duration during which an immunosuppressant therapy may be provided to the recipient will be reduced or eliminated. Additionally, because the effective amount of the calcineurin inhibitor to improve survival and function of the donor CD3+ cells and thereby engraftment of donor CD34+ cells is reduced in comparison to immunosuppressive amounts of the calcineurin inhibitor, the calcineurin inhibitor is not provided as an immunosuppressant.

The method of the present invention can be used to improve the survival and function of donor CD3+ cells in connection with engraftment of donor CD34+ cells and transplantation of any organ. For example, the organ may be a solid organ and may be selected from a group consisting of a heart, intestine, liver, lung, pancreas and kidney. Preferably, the solid organ is a kidney. In aspects of the invention the organ is a non-solid organ and may be selected from a group consisting of bone marrow, peripheral blood, and certain lymphoid tissue.

In aspects of the invention, the improved survival and function of the donor CD3+ cells facilitates mixed chimerism in the recipient by facilitating engraftment of the donor CD34+ cells and thereby allowing both donor and host myeloid lineages to exist in the host, followed by tolerization by intrathymic negative selection of the T-cells of the host or donor that would otherwise attach donor tissue or host tissue, respectively. In an aspect, the invention provides the administration of a calcineurin inhibitor together with cellular products in a recipient of an organ transplant from a donor. The CD3+ and CD34+ cells may be obtained from one or more apheresis products, which may be from a donor before or after they have already donated an organ. Preferably, the CD34⁺ cells are present in amount greater than 1×10⁴ CD34+ cells/kg recipient, and the CD3+ cells are present in an amount greater than 1×10⁴ CD3+ cells/kg recipient. Although, the skilled artisan will recognize that other CD34+ and CD3+ cell concentrations are within the scope of the invention, as exemplified throughout the application.

For example, the cellular products used herein may include various concentrations for each of the CD34⁺ cells and CD3⁺ cells, and different concentrations are discussed herein. The amount may be specified as a number of cells relative to the body mass of the recipient. For example, the cellular product may contain at least 1×10⁵, 2×10⁵, 5×10⁵, 1×10⁶, 2×10⁶, or 4×10⁶ CD34⁺ cells/kg recipient weight. The cellular product may contain at least 1×10⁴, 2×10⁴, 5×10⁴, 1×10⁵, 2×10⁵, 5×10⁵, 1×10⁶, 2×10⁶, 5×10⁶, 1×10⁷, 2×10⁷, 5×10⁷, or 1×10⁸ CD3⁺ cells/kg recipient weight.

Donor derived CD3+ and CD34+ cells may be obtained at any point after the subject has donated an organ. For example, an apheresis product may be obtained at least 1 day, at least 2 days, at least 3 days, at least 4 days, at least 5 days, at least 1 week, at least 2 weeks, at least 4 weeks, at least 8 weeks, at least 12 weeks, at least 24 weeks, at least 1 year, at least 2 years, or at least 5 years after the subject has donated a organ.

The solid organ may be any solid or non-solid organ that can be transplanted according to methods known in the art. For example and without limitation, the solid organ may be a kidney, lung, pancreas, pancreatic, islet cells, heart, intestine, colon, liver, skin, muscle, gum, eye, or tooth. Preferably, the solid organ is a kidney. In other aspects, the non-solid organ is bone marrow, peripheral blood, and certain lymphoid tissue. In certain embodiments, the invention is useful in treating brain or hematologic disorders. For example, an engraftment of CD34+ cells may be improved by administration or CD3+ cells together with the administration of a calcineurin inhibitor in order to produce functional cells of a hematopoietic lineage useful for the treatment of brain or hematologic disorders. In aspects of the invention, CD34+ cells may produce a lineage of microglial useful for the treatment of certain Central Nervous System diseases, such as brain disorders. In other aspects of the invention, engraftment of CD34+ cells may be improved by administration or CD3+ together with the administration of a calcineurin inhibitor in order to treat beta thalassemia or sickle cell anemia. In aspects of the invention, CD34+ cells may produce a lineage of normal blood cells useful for the treatment of beta thalassemia or sickle cell anemia.

The CD3⁺ cells, as well as the CD34+ cells may be HLA-matched to the recipient. The CD3⁺ cells and/or the CD34⁺ cells may be HLA-mismatched to the recipient. The donor and recipient may be HLA-matched at six, eight, ten, or twelve alleles among the HLA-A, HLA-B, HLA-C, HLA-DP, HLA-DQ, and HLA-DR genes. The donor and recipient may be HLA-mismatched at one, two, three, four, five, six, or more alleles among the HLA-A, HLA-B, HLA-C, HLA-DP, HLA-DQ, and HLA-DR genes.

In addition, the CD3⁺ cells, as well as the CD34+ and/or hFCs may be HLA-matched to the recipient. The CD3⁺ cells, CD34⁺ cells, and/or hFCs may be HLA-mismatched to the recipient. The donor and recipient may be HLA-matched at six, eight, ten, or twelve alleles among the HLA-A, HLA-B, HLA-C, HLA-DP, HLA-DQ, and HLA-DR genes. The donor and recipient may be HLA-mismatched at one, two, three, four, five, six, or more alleles among the HLA-A, HLA-B, HLA-C, HLA-DP, HLA-DQ, and HLA-DR genes.

The cells may be cryopreserved. The cells may contain one or more cryoprotectants. The cryoprotectant may be dextran having an average molecular weight of 40,000 Da or DMSO. The cellular product may contain the cryoprotectant at a concentration of about 1%, 2%, 3%, 4%, 5%, 7.5%, or 10%.

The cells may be provided in separate containers. The cells may be provided as a mixture in the same container.

The method may include any feature described above in relation to the compositions of the invention.

### DETAILED DESCRIPTION

The primary hurdle in organ transplantation is getting the recipient to tolerate the donor's tissue. If the recipient's immune system detects the donated organ as foreign, it attacks the tissue, leading to graft rejection. Consequently, most transplant recipients must take a combination of drugs that suppress the immune system. Immunosuppressive therapy, however, creates its own set of risks. To avoid graft rejection, transplantation of organs may be accompanied by transfer of donor derived CD3+ cells and CD34+ cells. Providing donor blood cells allows reconstitution of the recipient's immune system to include cells that have been educated to recognize the organ as non-foreign tissue. Consequently, the donated organ is not attacked, and the recipient tolerates the graft.

One strategy for reconstructing the recipient's immune system entails repopulating the recipient's immune system with donor-derived CD34+ cells. Successful engraftment of CD34+ cells allows for complete discontinuation of immunosuppression after the CD34+ cells are successfully engrafted.

However, CD34⁺ cells are relatively scarce, making up only about 0.1-0.2% of peripheral blood cells in normal, untreated patients, such as donors. Moreover, there are many transplant recipients for whom CD34+ engraftment was not achieved or was successful. Accordingly, there is a need for methods that improve the engraftment of donor CD34+ cells that can be collected. The methods of the present invention improve the efficacy of engrafting CD34+ cells in an organ donor recipient through the administration CD3+ cells together with calcineurin inhibitors. Calcineurin inhibitors have previously been known to be useful in specific dosages as an immunosuppressant, however by the present invention it has been discovered that at lower effective amounts calcineurin inhibitors improve the survival and function of CD3+ cells which improve the engraftment of donor derived hematopoietic cellular compositions comprising CD34+ cells.

### Cellular Products

All blood cells, including the cells of the immune system, are derived from hematopoietic stem cells (HSCs). HSCs are multipotent cells that can differentiate into various specialized cells and also reproduce to generate new HSCs. HSCs that differentiate form either lymphoid progenitors or myeloid progenitors. Lymphoid progenitors give rise to lymphocytes and natural killer cells. Myeloid progenitors produce cells of the myeloid and erythroid lineages, such as erythrocytes, platelets, basophils, neutrophils, eosinophils, monocytes, macrophages, and antigen-presenting cells, such as dendritic cells. In adults, most hematopoietic development occurs in the bone marrow, although maturation and activation of some lymphoid cells occurs in the spleen, thymus, and lymph nodes.

The cellular products of the invention include CD3+ positive cells and CD34+ cells. CD3+ and CD34+ cells may also be administered together with hematopoietic or human facilitating cells (hCFs). CD3+ cells and CD34+ cells are two populations of cells that allow donor HSCs to develop into mature cells of the immune system in the recipient's body.

CD3 comprises a group of polypeptides that interact with the two polypeptide chains of the T cell receptor to form the T cell receptor complex. The CD3 complex includes a gamma chain, delta chain, and two epsilon chains. CD3 is expressed on the surface of mature T cells and is thus useful as a marker for T cells.

CD34 is a cell surface marker that is expressed in HSCs and their immediate descendants, multipotent progenitor cells, which have not committed to either the myeloid or lymphoid lineage. Consequently, CD34 expression is a useful measure for identifying populations of cells that contain HSCs.

To promote engraftment of the CD34+ cells, cellular compositions are provided that include CD34+ cells and CD3+ cells in appropriate quantities. Compositions may also be provided that include hCFs in appropriate quantities. For example, an ample supply of CD3⁺ cells may promote engraftment of the CD34+ cells. It is also by the present invention understood that the administration of a calcineurin inhibitor improves survival and function of CD3+ cell, thereby further improving engraftment of CD34+ cells.

However, CD34+ cells are relatively scarce, making up only about 0.1-0.2% of peripheral blood cells in normal, untreated patients. Therefore, the cellular products may contain CD34+ cells that have been purified from an apheresis product to obtain a sufficient number of such cells. For example, the CD34+ cells may be purified using an immunomagnetic column system, as described below. In contrast, CD3+ cells are abundant, accounting for a majority of mononuclear cells in the peripheral blood. Thus, the population of CD3+ cells in the cellular products may be obtained from a portion of the apheresis product that has not been subjected to a column purification step. Alternatively or additionally, CD3+ cells may be obtained from a residual fraction following purification of CD34+ cells, such as the effluent of a column used to purify CD34+ cells.

CD3+ cells and CD34+ cells, and hFCs may be administered defined amounts. A useful unit of cell quantity in a product is the number of cells relative to the body mass of the recipient. For example and without limitation, the cellular product may contain at least 1×10⁴, 2×10⁴, 5×10⁴, 1×10⁵, 2×10⁵, 5×10⁵, 1×10⁶, 2×10⁶, or 4×10⁶, 1×10⁷, 2×10⁷, 4×10⁷, or 1×10⁸ CD34+ cells/kg recipient weight. For example and without limitation, the cellular product may contain at least 1×10⁴, 2×10⁴, 5×10⁴, 1×10⁵, 2×10⁵, 5×10⁵, 1×10⁶, 2×10⁶, 5×10⁶, 1×10⁷, 2×10⁷, 5×10⁷, or 1×10⁸ CD3⁺ cells/kg recipient weight. Other concentrations are exemplified in each of Strober et al., U.S. Pat. No. 9,504,717 and Strober et al., U.S. Pat. No. 9,561,253, the content of each of which is incorporated by reference herein in its entirety.

CD34⁺ cells administered may be at a designated level of purity. For example, the cellular product may contain CD34⁺ cells that are at least 30%, at least 40%, at least 50%, at least 60%, at least 70%, at least 80%, at least 90%, at least 95%, at least 98%, or at least 99% pure. Other purities are exemplified in each of Strober et al., U.S. Pat. No. 9,504,717 and Strober et al., U.S. Pat. No. 9,561,253, the content of each of which is incorporated by reference herein in its entirety.

The CD34⁺ cells and CD3⁺ cells may be derived from any subject that has donated an organ. The CD34⁺ cells and CD3⁺ cells may be from the same subject. The CD34⁺ cells and CD3⁺ cells may be from different subjects. Preferably, the CD34⁺ cells and CD3⁺ cells are derived from the subject that donated the organ that has been transplanted into the recipient.

Additionally, the hFCs and CD3⁺ cells may be derived from any subject that has donated an organ. The hFCs, CD34⁺ cells, and/or CD3⁺ cells may be from the same subject. The hFCs, CD34⁺ cells, and CD3⁺ cells may be from different subjects. Preferably, the hFCs , CD34⁺ cells, and/or CD3⁺ cells are derived from the subject that donated the organ that has been transplanted into the recipient.

The cellular composition may be provided in separate containers. The cellular compositions may be provided as a mixture in the same container.

The cells may be provided frozen. Consequently, the cells may contain a cryoprotectant. Any cryoprotectant known in the art may be used. For example and without limitation, the cryoprotectant may be DMSO, dextran having an average molecular weight of 40 kDa, serum, e.g., bovine serum, albumin, e.g., human serum albumin, or cell culture medium. The cryoprotectant may be present at a defined concentration. For example, the cellular product may contain about 1% DMSO, about 2% DMSO, about 5% DMSO, about 7.5% DMSO, about 10% DMSO, about 12.5% DMSO, about 15% DMSO, or about 20% DMSO. The cellular product may contain about 1% dextran, about 2% dextran, about 5% dextran, about 7.5% dextran, about 10% dextran, about 12.5% dextran, about 15% dextran, or about 20% dextran. Cyroprotection is discussed in each of Strober et al., U.S. Pat. No. 9,504,717 and Strober et al., U.S. Pat. No. 9,561,253, the content of each of which is incorporated by reference herein in its entirety.

The cells may be administered together with agents in addition to calcineurin inhibitors that enhance engraftment of the CD3+ cells. For example, agents may be administered that prevent a negative reaction of the recipient to the hematopoietic cells. For example and without limitation, the pharmaceutical composition may contain a cytokine, chemokine, growth factor, excipient, carrier, antibody or a fragment thereof, small molecule, drug, agonist, antagonist, matrix protein, or complementary cell type.

Compositions comprising CD34⁺ cells, CD3⁺ cells, and/or facilitating cells may contain a buffer. The cellular product may contain a buffer to maintain physiologically compatible pH. For example, the cellular product may be buffered to a neutral pH, such as from about 6.0 to about 8.0. When the cellular composition is provided in a composition in combination with a calcineurin inhibitor the pH should be compatible with both the cellular composition and the calcineurin inhibitor.

The cellular composition can be supplied in the form of a pharmaceutical composition, comprising an isotonic excipient prepared under sufficiently sterile conditions for human administration. Choice of the cellular excipient and any accompanying elements of the composition is adapted in accordance with the route and device used for administration. For general principles in medicinal formulation, see Cell Therapy: Stem Cell Transplantation, Gene Therapy, and Cellular Immunotherapy, by G. Morstyn & W. Sheridan. eds., Cambridge University Press, 1996; and Hematopoietic Stem Cell Therapy, E. D. Ball, J. Lister & P. Law, Churchill Livingstone, 2000.

The CD34⁺ cells, CD3⁺ cells, and/or hFCs may be HLA-matched or HLA-mismatched to the recipient. Human leukocyte antigens (HLAs), also called major histocompatibility complex (MHC) antigens, are protein molecules expressed on the surface of cells that confer a unique antigenic identity to these cells. MHC/HLA antigens are target molecules that are recognized by T-cells and natural killer (NK) cells as being derived from the same source of hematopoietic stem cells as the immune effector cells (“self”) or as being derived from another source of hematopoietic reconstituting cells (“non-self”). Two main classes of HLA antigens are recognized: HLA class I and HLA class II. HLA class I antigens (A, B, and C in humans) render each cell recognizable as “self,” whereas HLA class II antigens (DR, DP, and DQ in humans) are involved in reactions between lymphocytes and antigen presenting cells.

A key aspect of the HLA gene system is its polymorphism. Each gene exists in different alleles. Allelic gene products differ in one or more amino acids in the alpha and/or beta domain(s). An individual has two alleles of each gene, for a total of twelve alleles among the HLA-A, HLA-B, HLA-C, HLA-DP, HLA-DQ, and HLA-DR genes. An HLA-matched donor may have a match with the recipient at six, eight, ten, or twelve alleles selected from any combination of the HLA-A, HLA-B, HLA-C, HLA-DP, HLA-DQ, and HLA-DR genes. The genes most important for HLA typing are HLA-A, HLA-B, and HLA-DR, so the donor and recipient may be matched at all six alleles of the HLA-A, HLA-B, and HLA-DR genes. An HLA-mismatched donor may have a mismatch at one, two, three, four, five, six, or more alleles among the HLA-A, HLA-B, HLA-C, HLA-DP, HLA-DQ, and HLA-DR genes. HLA typing may be performed by any method known in the art. Examples of HLA typing methods include serological cytotoxicity, flow cytometry, and DNA typing. Such methods are described in, for example, U.S. Pat. No. 9,561,253, the contents of which are incorporated herein by reference.

The HLA genes are clustered in a super-locus present on chromosome position 6p21. Consequently, the set of alleles present on a single chromosome, i.e., a haplotype, tends to be inherited as a group. Identifying a patient's haplotypes can help predict the probability of finding matching donors and assist in developing a search strategy. Haplotypes vary in how common they are among the general population and in their frequency within different racial and ethnic groups.

Numerous exemplary embodiments are now described below, both HLA matched and HLA mismatched. The skilled artisan will recognize that the below embodiments are exemplary and non-limiting, particularly, the below embodiments do not limit any other part or exemplified cell amounts or combinations in any other part of this application.

Preparation of cellular products

As indicated above, CD34⁺ cells make up a low percentage of peripheral blood cells in normal subjects. However, the fraction of CD34⁺ cells in blood can be increased by administering to the subject a factor, such as granulocyte colony stimulating factor (G-CSF), that mobilizes CD34⁺ cells from bone marrow and other sources. Thus, prior to apheresis, the subject may be given G-CSF to mobilize CD34⁺ cells. Regimens for administering G-CSF to a subject prior to apheresis, including the dosage, frequency, and timing of administration, are known in the art and described in, for example, U.S. Pat. No. 9,561,253, the contents of which are incorporated herein by reference.

During preparation of the cellular products of the invention, cells may be frozen at any stage. For example, cells may be frozen immediately after an apheresis product is isolated from a donor but prior to separation into portions, after separation into portions, after purification or enrichment of CD34⁺ cells, or after combining purified CD34⁺ cells with CD3⁺ cells.

Cryopreservation of compositions of the invention may include addition of a cryoprotectant, such as a cryoprotectant described above. Cryopreservation typically involves reducing the temperature of the cell-containing sample at a controlled rate. Cryopreservation may include thawing the cell-containing sample and washing the sample to remove one or more cryoprotectants. Methods and reagents for cryopreservation, including freezing, thawing, and washing samples, are known in the art and described in, for example, U.S. Pat. No. 9,561,253, the contents of which are incorporated herein by reference.

CD34⁺ cells may be purified based on qualitative or quantitative expression of one or more cell surface markers. Examples of suitable cell surface markers include CD34, Thy-1, CD38, and AC133. CD34⁺ cells may be purified based on the presence or absence of a marker or on the level of expression of a marker, e.g., high vs. low.

CD34⁺ cells may be purified by selectively binding a suitable affinity reagent to CD34 or another marker. The affinity reagent may be an antibody, a full-length antibody, a fragment of an antibody, a naturally occurring antibody, a synthetic antibody, an engineered antibody, a full-length affibody, a fragment of an affibody, a full-length affilin, a fragment of an affilin, a full-length anticalin, a fragment of an anticalin, a full-length avimer, a fragment of an avimer, a full-length DARPin, a fragment of a DARPin, a full-length fynomer, a fragment of a fynomer, a full-length kunitz domain peptide, a fragment of a kunitz domain peptide, a full-length monobody, a fragment of a monobody, a peptide, a polyaminoacid, or the like. The affinity reagent may be directly conjugated to a detection reagent and/or purification reagent. The detection reagent and purification reagent may be the same, or they may be different. For example, the detection reagent and/or purification reagent may be fluorescent, magnetic, or the like. The detection reagent and/or purification reagent may be a magnetic particle for column purification, e.g., an immunomagnetic microsphere.

CD34⁺ cells may be isolated, enriched, or purified by any method. For example, CD34⁺ cells may be isolated, enriched, or purified by column purification, flow cytometery, cell sorting, or immunoadsorption column separation. Preferably, CD34⁺ cells are purified using an immunomagnetic column system, such as those sold under the trade name CliniMACS by Miltenyi Biotec Inc. (Auburn, CA), Methods of affinity purification of hematopoietic cells, including CD34⁺ cells, and analysis of purified populations are described in, for example, U.S. Pat. No. 9,561,253; U.S. Pat. No. 9,452,184; Ng et al., Isolation of human and mouse hematopoietic stem cells, Methods Mol Biol. (2009) 506:13-21. doi: 10.1007/978-1-59745-409-4_2; and Spohn et al., Automated CD34⁺ cell isolation of peripheral blood stem cell apheresis product, Cytotherapy (2015) October;17(10):1465-71. doi: 10.1016/j.jcyt.2015.04.005, the contents of each of which are incorporated herein by reference. The methods may include positive selection, negative selection, or both.

CD3⁺ cells may be obtained by dividing one or more apheresis products into two portions, using one portion to purify or enrich CD34⁺ cells, and using the second portion as a source of CD3⁺ cells. Alternatively, CD3⁺ cells may be obtained from a portion from which CD34⁺ cells have been purified, such as the effluent of column used to purify CD34⁺ cells, as described in, for example, U.S. Pat. No. 9,561,253, the contents of which are incorporated herein by reference.

CD34⁺ cells and/or CD3⁺ cells may be expanded ex vivo. Expansion may occur prior to, or subsequent to, freezing. Expansion may include providing one or more growth factors, and it may include culturing cells in the presence of another cell type, e.g., feeder cells. Methods for expanding hematopoietic cells are described in, for example, U.S. Pat. No. 9,561,253, the contents of which are incorporated herein by reference.

### Providing the Regimen of the Present Invention

The methods of the invention comprise administering cellular compositions including CD3+ and CD34+ cells and a calcineurin inhibitor to a recipient of an organ transplant. The cellular compositions and calcineurin inhibitor may be provided by any suitable means. For example and without limitation, the cellular compositions and/or calcineurin inhibitors may be delivered to the recipient by injection using a needle, catheter, central line or the like. In some cases, the cells, calcineurin inhibitors, and/or compositions, may be delivered intravascularly, intravenously, intraarterially, subcutaneously, intramuscularly, directly to the bone, or through any source which permits the hematopoietic cells to home to an appropriate site in the recipient such that the hematopoietic cells persist, regenerate and differentiate in the recipient. The cellular compositions and/or calcineurin inhibitor may be provided by infusion. Administration of the cellular compositions and/or calcineurin inhibitor may be provided in an inpatient procedure or in an outpatient procedure. An inpatient procedure requires admission to a hospital, and the patient may spend one or more nights in the hospital. An outpatient procedure does not require admission to a hospital and may be performed in a non-hospital setting, such as a clinic, doctor's office, home, or other location.

Methods of the present invention may be used in conjunction with transplantation of any solid or non-solid organ. For example and without limitation, the solid organ may be a kidney, lung, pancreas, pancreatic, islet cells, heart, intestine, colon, liver, skin, muscle, gum, eye, or tooth. In aspects of the invention, without limitation the non-solid organ may be bone marrow, peripheral blood, and lymphoid tissue. The transplant may include a complete organ, a portion of an organ, or cells from a tissue of an organ. The cellular product may be provided prior to, during, or subsequent to the organ transplant. For example and without limitation, the cellular product may be provided one, two, three, four, five, or six days or one, two, three, or four weeks prior to the organ transplant, or it may be provided one, two, three, four, five, or six days or one, two, three, or four weeks after the organ transplant.

To facilitate survival and function of CD3+ cells in the recipient and thereby engraftment of CD34+ improved by the presence of the CD3+ cells, in addition to the administration of calcineurin inhibitors, the recipient's immune system may be conditioned in conjunction with providing the cellular product. For example, non-myeloablative conditioning may be used. In non-myeloablative conditioning, the recipient is exposed to drugs, antibodies, irradiation, or some combination thereof at a dose that is too low to eradicate all the bone marrow cells. Typically, the conditioning regimen includes treatment with anti-thymocyte globulin (ATG), total lymphoid irradiation, and corticosteroids (e.g. prednisone) for a period of from about 10 to 12 days (e.g. for about 11 days). The irradiation may be targeted to a particular location of the recipient's body. For example, irradiation may be targeted to a tissue, an organ, a region of the body or the whole body. Irradiation may be targeted to the lymph nodes, the spleen, or the thymus or any other area known to a person of skill in the art. When multiple doses of irradiation are administered, the doses may be targeted to the same location or to different locations. Non-myeloablative conditioning may include the use of a T cell depleting agent, such as a monoclonal antibody or drug, e.g., fludarabine.

Regimens for non-myeloablative conditioning are known in the art and are described in, for example, U.S. Pat. No. 9,561,253, the contents of which are incorporated herein by reference.

The methods may include immunosuppressive therapy. Advantageously, because the administration of the calcineurin inhibitors increases the survival and efficacy of CD3+ cells in the recipient and thereby engraftment of CD34+ cells, the duration and/or the amount of immunosuppressive therapies can be reduced. Immunosuppressive therapy, or immunosuppression, involves treatment of the graft recipient with agents that diminish the response of the host immune system against the donor cells, which can lead to graft rejection. Because the regimens and compositions of the present invention result in improved survival and function of the CD3+ cells, engraftment of CD34+ cells is improved and the need for immunosuppressive therapy is greatly reduced. These immunosuppressive therapies primary immunosuppressive agents include antibody-based therapies, such as use monoclonal (e.g., muromonab-CD3) or polyclonal antibodies or anti-CD25 antibodies (e.g., basiliximab, daclizumab). Antibody-based therapy allows for avoidance or dose reduction of calcineurin inhibitors, possibly reducing the risk of nephrotoxicity. Regimens for immunosuppressive therapy are known in the art and are described in, for example, U.S. Pat. No. 9,561,253, the contents of which are incorporated herein by reference.

Immunosuppression may also diminish the response of the donor immune cells against recipient tissue, which can lead to GVHD. GVHD may be acute or chronic. Acute GVHD typically occurs in the first 3 months after graft and may involve the skin, intestine, or the liver. Treatment for acute GVHD usually includes high-dose corticosteroids such as prednisone. Chronic GVHD typically occurs after the first 3 months following transplant and is the major source of late treatment-related complications. Chronic GVHD may cause functional disability and require prolonged immunosuppressive therapy.

Immunosuppressive therapy may occur in multiple phases. For example, the immunosuppressive regimen may have an induction phase and a maintenance phase. Induction and maintenance phase strategies may use different medicines at doses adjusted to achieve target therapeutic levels to enhance engraftment of the CD34+ cells in the recipient.

Immunosuppressive therapy may be withdrawn after engraftment of the CD34+ cells has been established in the recipient. Advantageously, the regimens and compositions of the present invention greatly reduce the need to immunosuppressive therapies following organ donation and allow for immunosuppressive therapies to be withdrawn quickly after organ transplantation due to the establishment of engrafted CD34+ cells in the in the recipient. The CD34+ cell engraftment status of the recipient may be monitored as described below and deemed stable after a certain period, for example, 3 months, 6 months 12 months, 18 months, 24 months, or longer. Thus, immunosuppression may be discontinued for the recipients after a certain period, for example, 3 months, 6 months 12 months, 18 months, 24 months, or longer. Withdrawal of immunosuppressive therapy may include tapering, i.e., progressively reducing the dosage or frequency of treatment.

The present invention may also be utilized to engraft CD34+ cells in a manner that produces mixed chimerism in the recipient. A determination of whether an individual is a full chimera, mixed chimera, or non-chimera made be made by an analysis of a hematopoietic cell sample from the organ transplant recipient, e.g. peripheral blood, bone marrow, etc. as known in the art. Analysis may be done by any convenient method of typing. Analysis may be performed on hematopoietic cells or a subset thereof, such as all mononuclear cells, T cells, B cells, CD56⁺TK cells, and CD15⁺ neutrophils. Chimerism can be assessed by PCR analysis of microsatellites. For example, commercial kits that distinguish polymorphisms in short terminal repeat lengths of donor and host origin are available. Automated readers provide the percentage of donor type cells based on standard curves from artificial donor and host cell mixtures.

Recipients may be categorized as fully chimeric, mixed chimeric, or non-chimeric based on the fraction of cells that are derived from the donor. For example, recipients can be deemed fully chimeric if they have at least 90%, at least 95%, at least 98%, or at least 99% donor-derived cells. Recipients can be deemed mixed chimeric if they have too few donor-derived cells to be categorized as fully chimeric but a fraction of donor-derived cells that exceeds a certain threshold, such as at least 0.5%, at least 1%, at least 2%, at least 3%, at least 5%, at least 7.5%, at least 10% donor-derived cells. Recipients can be deem non-chimeric if the fraction of donor-derived cells falls below the threshold required to be categorized as mixed chimeric.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof

What is claimed is:

1. A method for transplantation of an organ from a donor, the method comprising:
implanting an organ from a donor in a recipient;
administering to the recipient a composition comprising donor derived CD3+ cells and CD34+ cells; and
administering to the recipient a regimen comprising a calcineurin inhibitor in an effective amount to improve survival and/or function of the donor CD3+ cells in the recipient, thereby enabling the donor CD3+ cells to better facilitate engraftment of the donor CD34+ cells in the recipient.

2. The method of claim 1, wherein the effective amount of the calcineurin inhibitor provided to improve survival and/or function of the donor CD3+ cells in the recipient is lower than an amount provided for immune suppression therapy.

3. The method of claim 1, wherein the composition comprises at least 1×10⁴ CD3⁺ cells/kg recipient weight.

4. The method of claim 1, wherein the composition comprises at least 1×10⁴ CD34⁺ cells/kg recipient weight.

5. The method of claim 4, wherein the composition further comprises human facilitating cells (hFCs).

6. The method of claim 5, wherein the hFCs express one or more of the phenotypes selected from CD8+/alpha beta TCR−/CD56^{dim/meg} and CD8+/alpha beta TCR−/CD56^{bright} cells.

7. The method of claim 1, wherein the calcineurin inhibitor is selected from group consisting of cyclosporine A, tacrolimus, and sirolimus.

8. The method of claim 1, wherein the effective amount of the calcineurin inhibitor is administered according to at least one selected from the group consisting of: simultaneously with the composition comprising donor derived CD3+ cells and CD34+ cells, prior to administration of the composition comprising donor derived CD3+ cells and CD34+ cells , after administration of the composition comprising donor derived CD3+ cells and CD34+ cells, and a combination thereof.

9. The method of claim 8, wherein the effective amount of the calcineurin inhibitor is administered at or less than about 1 month prior to administration of the composition comprising donor derived CD3+ cells and CD34+ cells .

10. The method of claim 8, wherein the effective amount of the calcineurin inhibitor is administered at or more than about 1 day after administration of the composition comprising donor derived CD3+ cells and CD34+ cells.

11. The method of claim 1, wherein the regimen comprising an effective amount of the calcineurin inhibitor is administered for at least about 1 day.

12. The method of claim 1, wherein the organ is a solid organ is selected from a group consisting of a heart, intestine, liver, lung, pancreas and kidney.

13. The method of claim 12, wherein the solid organ is a kidney.

14. The method of claim 1, wherein the organ is a non-solid organ selected form the group consisting of bone marrow, peripheral blood, and lymphoid tissue

15. The method of claim 14, wherein the non-solid organ is bone marrow.

16. The method of claim 1, wherein the method facilitates establishing mixed chimerism in the recipient.
